(19) United States
(12) Patent Application Publication
(10) Pub. No.: US 2026/0295193 A1
(43) Pub. Date: Oct. 1, 2026

(54) CONDUCTIVE TEXTILE TUBE OR CONDUIT

(71) Applicant: ResMed Pty Ltd, Bella Vista, New South Wales (AU)

(72) Inventors: Michael James DENT, Sydney (AU); Lina GIRDAUSKAITE, Sydney (AU); Samuel Thomas HORLER, Sydney (AU); Skye Kimberley SHARMA, Sydney (AU); Ting Lee TEH, Sydney (AU); Chiew Khuen WONG, Sydney (AU)

(21) Appl. No.: 19/475,813

(22) PCT Filed: Apr. 22, 2024
(86) PCT No.: PCT/AU2024/050381

§ 371 Date: Oct. 16, 2025

(30) Foreign Application Priority Data
Apr. 21, 2023 (AU) 2023901185

(51) Int. Cl. A61M 16/08; A61M 16/00; A61M 16/10

(52) U.S. Cl. CPC A61M 16/0875; A61M 16/0003; A61M 16/1095; A61M 2016/0033; A61M 2205/0233; A61M 2205/0238; A61M 2205/3331; A61M 2205/3368; A61M 2205/3653; A61M 2207/00

(57) ABSTRACT

An air conduit for a system for treating a respiratory disorder comprises a textile outer layer and at least one inner layer comprising a polymer layer. The outer textile layer is a conductive textile and/or at least one of the inner layers comprises a conductive coating. The conduit may form part of an air circuit and/or a headgear of a patient interface. An air conduit with an electrically conductive intermediate layer is also disclosed.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

In many examples of the prior art an air circuit may comprise one or more heating elements configured to heat air in the air circuit, for example to maintain or raise the temperature of the air. The heating element may be in a form of a heated wire circuit (typically copper wire), and may comprise one or more transducers, such as temperature sensors. In one form, the heated wire circuit may be helically wound around the axis of the air circuit. The heating element may be in communication with a controller such as a central controller of an RPT device. One example of an air circuit comprising a heated wire circuit is described in U.S. Pat. No. 8,733,349, which is incorporated herewithin in its entirety by reference.

While such heating elements may function well to prevent condensation within the air circuit (so called “rainout”), they may add to the weight of the air circuit, and may add to the complexity of manufacturing the air circuit.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramactrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a tube comprising a textile outer layer and at least one inner layer comprising a polymer layer, wherein the outer textile layer is a conductive textile and/or at least one of the inner layers comprises a conductive coating.

One form of the present technology comprises an air conduit for a system for treating a respiratory disorder, the air conduit comprising a textile outer layer and at least one inner layer comprising a polymer layer, wherein the outer textile layer is a conductive textile and/or at least one of the inner layers comprises a conductive coating.

In examples:
a) the textile layer comprises at least one conductive fibre;
b) the textile layer is formed by circular knitting or weaving and at least one of the weft yarns is a conductive fibre;
c) the textile layer is formed by conventional or circular knitting and at least one of the warp yarns is a conductive fibre;
d) the textile is formed from a braiding process and at least one of the yarns is a conductive fibre;
e) the polymer layer comprises at least one track formed from a conductive ink, paste or gel or a liquid metal;
f) the air conduit comprises a further inner layer, between the polymer layer and the textile layer;
g) the further inner layer comprises a conductive element;
h) the conductive element comprises a flexible printed circuit board (PCB), one or more wires or a sheet of conductive material;
i) at least one of the inner layer(s) and/or conductive textile is configured to conduct electrical signals;
j) at least one of the inner layer(s) and/or conductive textile is configured to conduct electrical power;
k) at least one of the inner layer(s) and/or conductive textile is configured to heat the air conduit when conducting an electrical current;
l) the air conduit comprise at least one sensor and/or at least one antenna, wherein the at least one sensor and/or at least one antenna are electrically connected to the conductive textile and/or conductive coating;
m) the air conduit forms part of an air circuit; and/or
n) the air conduit forms part of a headgear for a patient interface.

Another form of the present technology comprises an air conduit for a system for treating a respiratory disorder, the air conduit comprising a textile outer layer, at least one inner layer comprising a polymer layer, and at least one electrically conductive intermediate layer.

Preferably:
a) the intermediate layer comprises a flexible printed circuit board (PCB), one or more wires and/or a sheet of conductive material;
b) the electrically conductive intermediate layer is configured to conduct electrical signals;
c) the electrically conductive intermediate layer is configured to conduct electrical power;
d) the electrically conductive intermediate layer is configured to heat the air conduit when conducting an electrical current;
e) the air conduit comprises at least one sensor and/or at least one antenna, wherein the at least one sensor and/or at least one antenna are electrically connected to the electrically conductive intermediate layer;
f) the air conduit forms part of an air circuit; and/or
g) wherein the air conduit forms part of a headgear for a patient interface.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

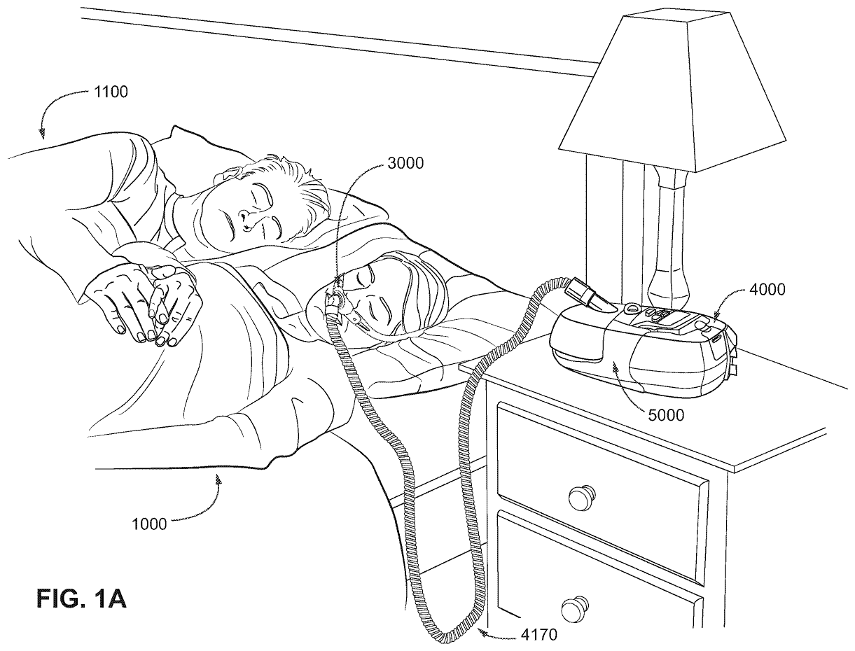

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

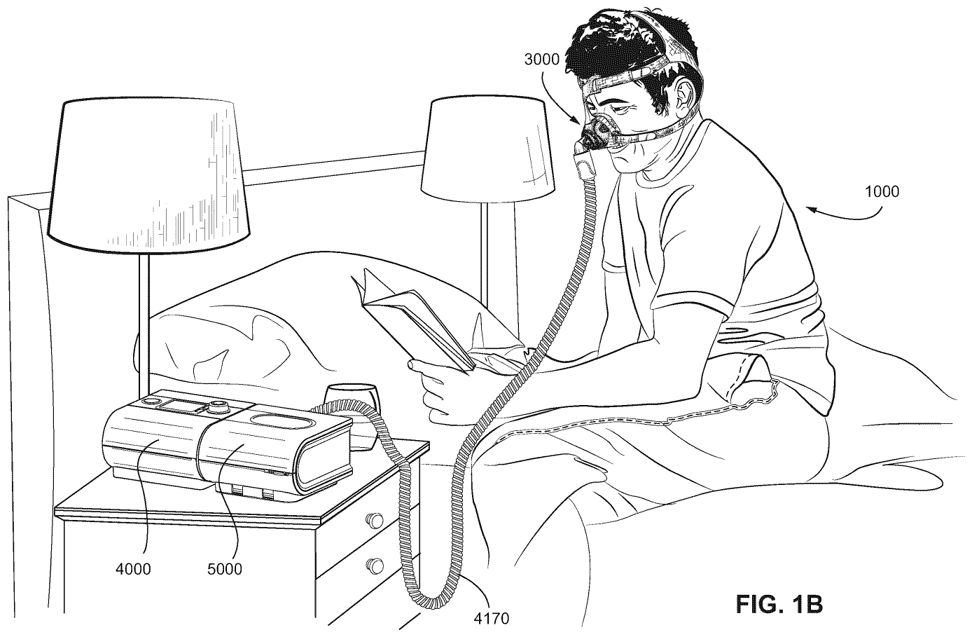

FIG. 2 shows an overview of a human respiratory system including the nasal and oral cavities, the larynx, vocal folds, oesophagus, trachea, bronchus, lung, alveolar sacs, heart and diaphragm.

### 3.3 Patient Interface

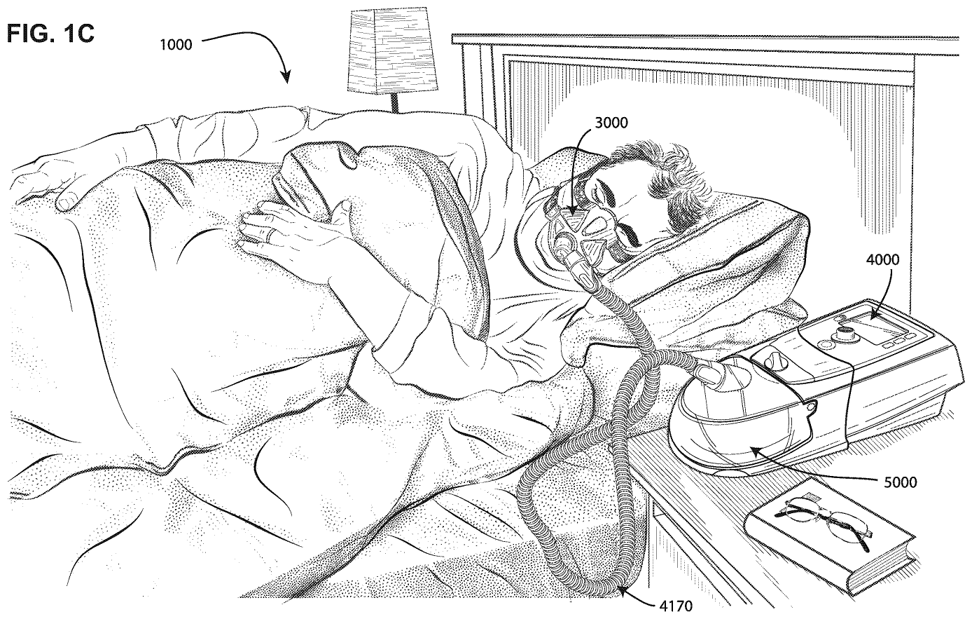

FIG. 3A shows a patient interface in the form of a nasal mask in accordance with one form of the present technology.

FIG. 3B shows a patient interface having conduit headgear, in accordance with one form of the present technology.

### 3.4 Air Conduit

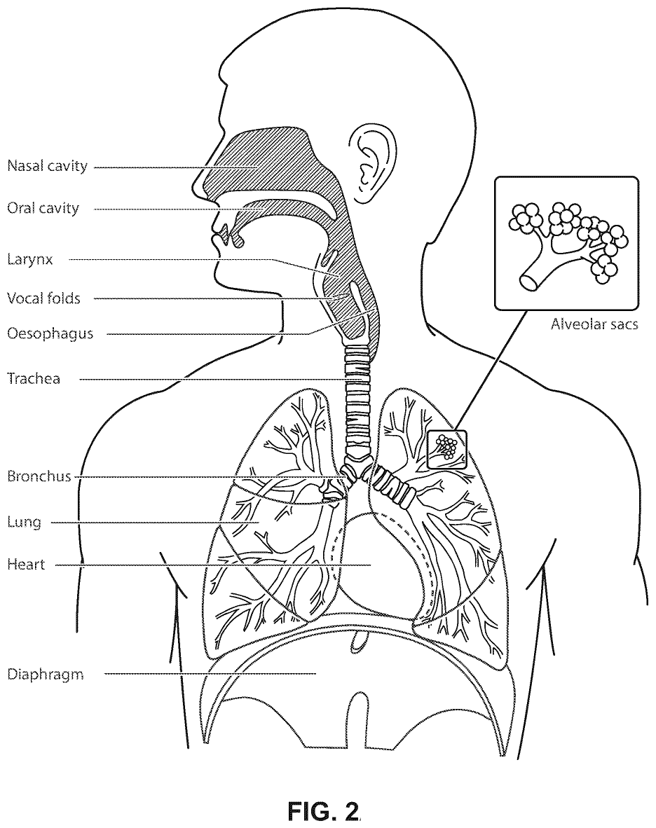

FIG. 4 is a diagrammatic side view of a portion of an air conduit of the present technology, with an inner polymer layer shown in hidden detail.

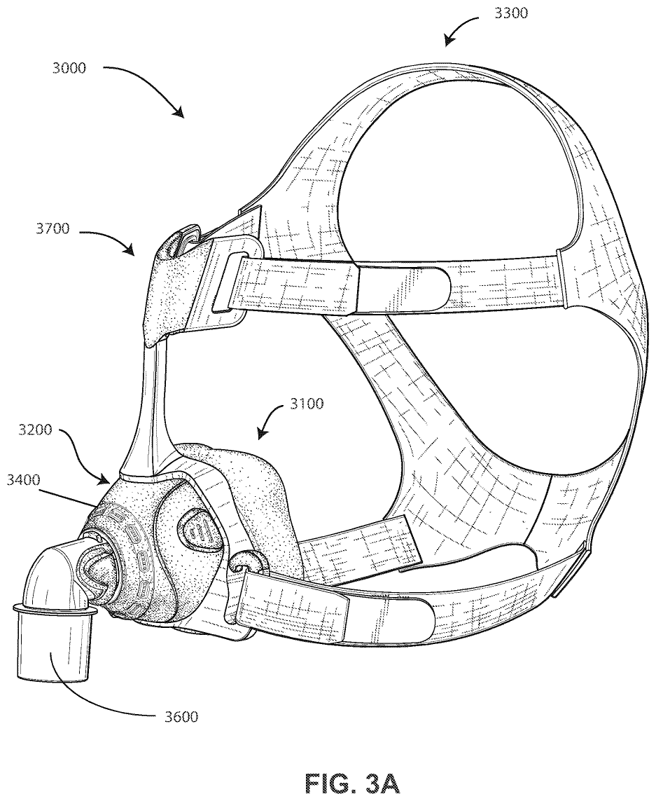

FIG. 5 is a diagrammatic view of a portion of a knitted conductive textile.

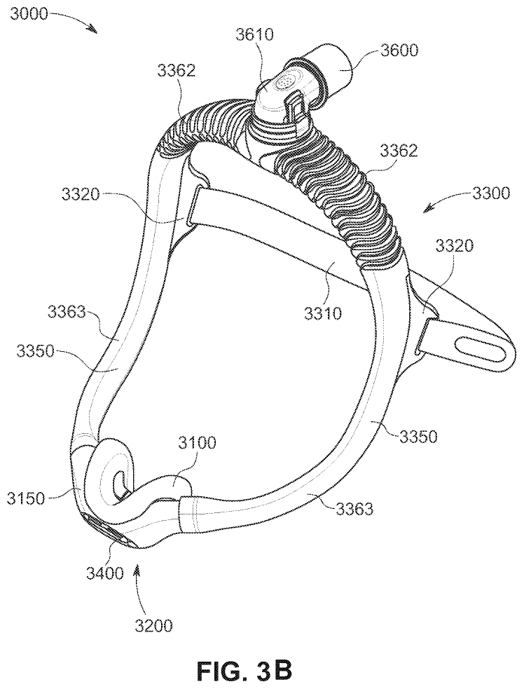

FIG. 6 is a diagrammatic view of a portion of a woven conductive textile.

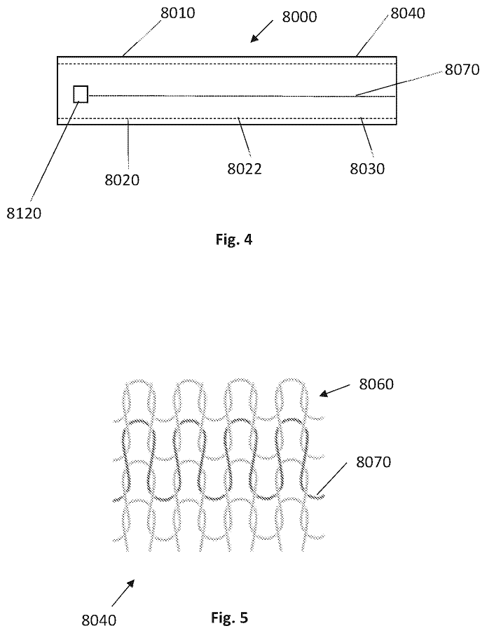

FIG. 7 is a diagrammatic view of a portion of a braided conductive textile.

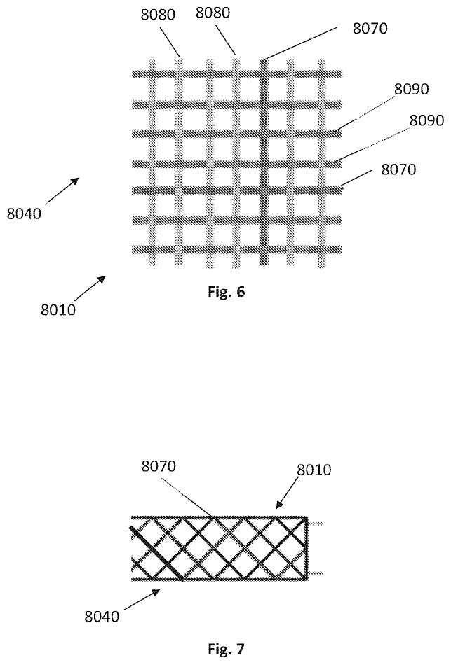

FIG. 8 is a diagrammatic cross-section view of one form of an air conduit of the present technology.

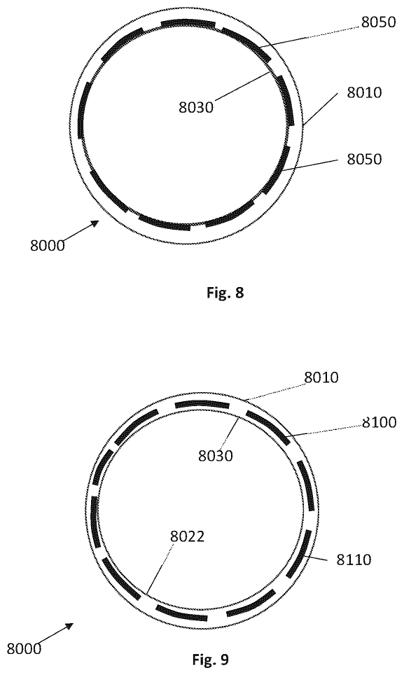

FIG. 9 is a diagrammatic cross-section view of another form of an air conduit of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3B, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components (e.g., a cushion module 3150 together with the seal-forming structure 3100) in the sense that it or they can be replaced with different components, for example components of a different size.

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3B, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3B, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes. The tubes 3350 may comprise tabs 3320 for connecting a back strap 3310.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3B, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet for receiving the flow of pressurized air.

Each tube 3350 may be configured to receive a flow of air from the connection port 3600 on top of the patient's head and to deliver the flow of air to the seal-forming structure 3100 at the entrance of the patient's airways. In the example shown in FIG. 3B, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

Conduits forming part of the positioning and stabilising structure 3300, like headgear straps, may provide a force that contributes to the positioning and stabilising force. In some forms, the conduits may provide a force directed into the patient's head when the conduits are filled with pressurized air. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3B comprises tubes 3350, the superior portions of which comprise extendable tube sections each in the form of an extendable concertina structure 3362.

In some forms of the technology one or both of the tubes 3350 may be formed wholly or partially by an air conduit which is capable of conducting electricity, as described further below.

### 4.4 RPT Device

An RPT device 4000 in accordance with one aspect of the present technology comprises mechanical, pneumatic, and/or electrical components and is configured to execute one or more algorithms such as any of the methods, in whole or in part, described herein. The RPT device 4000 may be configured to generate a flow of air for delivery to a patient's airways, such as to treat one or more of the respiratory conditions described elsewhere in the present document.

### 4.5 Air Conduit

Referring next to FIG. 4, in one form of the technology a tube, e.g. an air conduit 8000, which may, for example, form part of an air circuit 4170 and/or a positioning and stabilising structure 3300 for a patient interface 3000, is capable of conducting electricity (at power voltages and currents and/or at signal voltages and currents) over at least part of its length. For example, the air conduit 8000 may form, in whole or in part, a tube 3350 of a positioning and stabilising structure 3300 of a patient interface 3000 such as that shown in FIG. 3B or the air circuit 4170 such as that shown in FIG. 1C.

According to one form of the technology, the air conduit 8000 comprises a textile outer layer 8010 and at least one inner layer 8020 comprising a polymer layer 8030. In examples the innermost layer 8022 is a polymer layer 8030 which is substantially air impermeable.

In examples, the textile outer layer 8010 is a conductive (e.g. electrically conductive) textile. In other examples at least one of the inner layers 8020 comprises an electrically conductive coating 8050 (see FIG. 8). In some forms of the technology the outer textile layer is a conductive textile 8040 and one of the inner layers 8020 comprises a conductive coating 8050.

In one form of the technology the conductive coating 8050 and/or the conductive textile 8040 is configured to heat the air conduit 8000, or at least the innermost layer 8022, when conducting an electrical current. Additionally, or alternatively, the conductive coating 8050 and/or the conductive textile 8040 may be configured to transmit power between components of a system for treating a respiratory disorder (e.g. from an RPT device to an actuator or valve) and/or to transmit electrical signals between such components, as described further below.

The textile outer layer 8010 may be permanently bonded to the polymer layer 8030, or it may take the form of a removable cover.

The removable cover may also consist of the textile outer layer 8010 with a conductive coating 8050. In other examples the conduit may comprise a removable cover comprising a textile outer layer and conductive coating 8050 and an inner polymer layer 8330 or inner textile layer.

### 4.5.1 Conductive Textile

In examples, conductive threads or fibres may form part of the textile which forms part of the air conduit 8000.

The textile may formed by knitting (including circular knitting or 3D knitting), weaving, braiding, or any similar process.

For example, as shown in FIG. 5, the textile outer layer 8010 may be a conductive textile 8040, e.g. a knitted textile 8060. Highly deformable conductive fibres 8070 (for example threads comprised of mixed conductive and non-conductive fibres with mechanical properties suitable for knitting process) may be knitted into the textile 8060, such that they form part of the structure of the textile 8060. In another example (not shown) conductive fibres may be interwoven into a knitted textile without forming part of the textile structure (e.g. without forming part of a course of the textile 8060). This may be particularly suitable if the conductive fibres 8070 have a relatively low flexibility (for example purely metallic monofilament fibres from steel, titanium, aluminium, silver, gold or copper, with thin diameters from 1 to 80 μm) as their mechanical properties may not allow using them in knitting process.

The conductive fibres 8070 may comprise conductive polymers, for examples one or more of polyacetylene, polypyrrole, and polyaniline. The conductive fibres 8070 may also comprise suitable metals and/or non-conductive materials which are coated with a suitably conductive coating (e.g. carbon nanotubes or metallic particles).

Referring next to FIG. 6, in one example, the textile layer 8010 may be formed from a woven textile comprising warp threads 8080 and weft threads 8090. At least one of the warp and weft threads 8080, 8090 may comprise conductive fibres 8070.

Referring next to FIG. 7, in another example the textile layer 8010 may be braided. At least one conductive fibre 8070 may be incorporated into the textile layer 8010.

In other examples of the technology, conductive textile fibres 8070 may be provided to a surface of the textile layer 8010 (e.g. the inner surface). In one example, a conductive portion, for example a conductive fibre 8070, maybe applied to a surface of the textile layer 8010 by an embroidery process. In another example, a conductive fibre 8070 may be attached to the surface of the textile layer 8010 by a tailored fibre placement (TFP) process, wherein a roving material comprising conductive fibres is stitched to a surface of the textile.

### 4.5.2 Polymer Layer

Referring next to FIG. 8, in examples a conductive coating 8050 (or a plurality of such coatings) may be provided to the exterior surface of the polymer layer 8030 by means of a conductive ink, paste, liquid metal gel or similar. In one example screen printing is used to deposit the conductive material. In other examples the conductive coating 8050 may comprise metallic and/or carbon particles.

The conductive coating 8050 preferably defines a conductive track (or a plurality of such tracks) on the surface of the polymer layer 8030, rather than coating the entire surface of the polymer layer 8030.

### 4.5.3 Intermediate Layer

Referring next to FIG. 9, in examples, the air conduit 8000 may comprise one or more intermediate inner layers 8100 inside the outer textile layer 8010 (e.g. radially inside) but outside the polymer layer 8030. At least one of the intermediate layers 8100 may be capable of conducting electricity. In one form of the technology an intermediate layer comprises a flexible printed circuit board 8110 comprising at least one conductive track. In other forms of the technology the intermediate layer may comprise an alternative conductive element such as one or more wires and/or a sheet of conductive material.

### 4.5.4 Helical Track

In examples which are configured to heat the air conduit 8000, at least one conductive portion of the air conduit 8000 (e.g. conductive fibres 8070 or conductive surface coatings 8050) may be configured in a generally helical path around the air conduit 8000. In other examples, the conductive portion may be orientated substantially longitudinally along the length of the conduit.

### 4.5.5 Electrical/Electronic Components

Referring back to FIG. 4, the air conduit 8000 may comprise a suitable electrical component 8120, for example a flow sensor, humidity sensor and/or a temperature sensor (e.g. thermocouple, platinum resistance thermometer or thermistor, etc). Further details of such components are disclosed in U.S. Patent Application Publication 2008/0105257 A1, the contents of which are incorporated herein by reference. Such electrical components may be in electrical communication with a controller (e.g. a controller of an RPT device and/or a humidifier) via the air conduit 8000, e.g. via one or more conductive fibres 8070.

In examples, the air conduit 8000 may further comprise one or more antennas, for example a Bluetooth antenna, Wi-Fi antenna and/or an RFID antenna. Such antennas may also be in electrical communication with a controller (e.g. a controller of an RPT device and/or a humidifier) via the air conduit 8000, e.g. via one or more conductive fibres 8070.

### 4.6 Air Circuit

An air circuit 4170 in accordance with an aspect of the present technology is a conduit or a tube (for example the conductive tube described above) constructed and arranged to allow, in use, a flow of air to travel between two components such as RPT device 4000 and the patient interface 3000.

In particular, the air circuit 4170 may be in fluid connection with the outlet of the pneumatic block of an RPT device and the patient interface. The air circuit may be referred to as an air delivery tube. In some cases there may be separate limbs of the circuit for inhalation and exhalation. In other cases a single limb is used.

### 4.6.1 Supplementary Gas Delivery

In one form of the present technology, supplementary gas, e.g. oxygen, is delivered to one or more points in the pneumatic path, such as upstream of the pneumatic block, to the air circuit 4170, and/or to the patient interface 3000.

### 4.7 Humidifier

### 4.7.1 Humidifier Overview

In one form of the present technology there is provided a humidifier 5000 (e.g. as shown in FIG. 1A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

### 4.8 Glossary

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

4.10 REFERENCE SIGNS LIST

| | |
|---|---|
| patient | 1000 |
| bed partner | 1100 |
| patient interface | 3000 |
| seal - forming structure | 3100 |
| cushion module | 3150 |
| positioning and stabilising structure | 3300 |
| strap | 3310 |
| tab | 3320 |
| tube | 3350 |
| concertina structure | 3362 |
| non - extendable tube sections | 3363 |
| vent | 3400 |
| connection port | 3600 |
| elbow | 3610 |
| forehead support | 3700 |
| RPT device | 4000 |
| air circuit | 4170 |
| humidifier | 5000 |
| air conduit | 8000 |
| textile layer | 8010 |
| inner layer | 8020 |
| innermost layer | 8022 |
| polymer layer | 8030 |
| conductive textile | 8040 |
| conductive coating | 8050 |
| knitted textile | 8060 |
| conductive fibre | 8070 |
| warp threads | 8080 |
| weft threads | 8090 |
| intermediate layer | 8100 |
| flexible PCB | 8110 |
| component | 8120 |

1.-22. (canceled)

23. An air conduit for a system for treating a respiratory disorder, the air conduit comprising a textile outer layer and at least one inner layer comprising a polymer layer, wherein the outer textile layer is a conductive textile and/or at least one of the inner layers comprises a conductive coating.

24. The air conduit of claim 23, wherein the textile layer comprises at least one conductive fibre.

25. The air conduit of claim 23, wherein the textile layer is formed by circular knitting or weaving and at least one of the weft yarns is a conductive fibre.

26. The air conduit of claim 23, wherein the textile layer is formed by conventional or circular knitting and at least one of the warp yarns is a conductive fibre.

27. The air conduit of claim 23, wherein the textile outer layer is formed from a braiding process and at least one of the yarns is a conductive fibre.

28. The air conduit of claim 23, wherein the polymer layer comprises at least one track formed from a conductive ink, paste or gel or a liquid metal.

29. The air conduit of claim 23, wherein at least one of the inner layers comprises a conductive element.

30. The air conduit of claim 29, wherein the conductive element comprises a flexible printed circuit board (PCB).

31. The air conduit of claim 29, wherein the conductive element comprises one or more wires.

32. The air conduit of claim 29, wherein the conductive element comprises a sheet of conductive material.

33. The air conduit of claim 23, wherein at least one of the inner layer(s) and/or conductive textile is configured to conduct electrical signals.

34. The air conduit of claim 23, wherein at least one of the inner layer(s) and/or conductive textile is configured to conduct electrical power.

35. The air conduit of claim 23, wherein at least one of the inner layer(s) and/or conductive textile is configured to heat the air conduit when conducting an electrical current.

36. The air conduit of claim 23, wherein the air conduit comprises at least one sensor and/or at least one antenna.

37. The air conduit of claim 36, wherein the at least one sensor and/or at least one antenna are electrically connected to the conductive textile and/or conductive coating.

38. The air conduit of claim 23, further comprising at least one electrically conductive intermediate layer and at least one sensor and/or at least one antenna electrically connected to the intermediate layer.

39. The air conduit of claim 23, wherein the air conduit forms part of an air circuit.

40. The air conduit of claim 23, wherein the air conduit forms part of a headgear for a patient interface.
